(19) United States
(12) Patent Application Publication
(10) Pub. No.: US 2014/0275502 A1
(43) Pub. Date: Sep. 18, 2014

(54) PROCESS FOR THE PREPARATION OF CERTAIN TRIARYL RHAMNOSE CARBAMATES

(71) Applicant: Dow AgroSciences LLC, Indianapolis, IN (US)

(72) Inventors: Natalie C. GIAMPIETRO, Carmel, IN (US); Lawrence C. CREEMER, Greenfield, IN (US); Gary D. CROUSE, Noblesville, IN (US)

(73) Assignee: Dow AgroSciences LLC, Indianapolis, IN (US)

(21) Appl. No.: 14/192,442

(22) Filed: Feb. 27, 2014

Related U.S. Application Data
(60) Provisional application No. 61/778,472, filed on Mar. 13, 2013.

(51) Int. Cl. C07H 1/00; C07D 249/12; C07D 249/08; C07H 23/00

(52) U.S. Cl. CPC C07H 1/00; C07H 23/00; C07D 249/12; C07D 249/08
USPC 536/17.1; 536/17.4; 548/263.2; 548/265.8

(57) ABSTRACT

Aryl boronic esters and boronic acids containing the rhamnose carbamate moiety are coupled to a triazole with an appropriate leaving group, generating a 4-triazolylphenyl carbamate in good yield and without cleavage of the carbamate linkage.

### CROSS REFERENCE TO RELATED APPLICATIONS

This application claims the benefit of U.S. Provisional Patent Application Ser. No. 61/778,472 filed Mar. 13, 2013, the entire disclosure of which is hereby expressly incorporated by reference.

### BACKGROUND OF THE INVENTION

The present invention concerns an improved process for preparing certain triaryl rhamnose carbamates.

WO 2009102736 (A1) describes, inter alia, certain triaryl rhamnose carbamates and their use as insecticides. One of the methods used to prepare such triaryl compounds is by way of a Suzuki coupling reaction, wherein an aryl boronic acid or ester is coupled with a halogenated heterocycle. However, due to the lability of the carbamate linkage during the Suzuki process, the examples in WO 2009102736 (A1) are devoid of precursors that contain the sugar-carbamate moiety. It would be desirable to have a process in which aryl boronic esters and boronic acids containing the rhamnose carbamate moiety can be coupled to a triazole with an appropriate leaving group, generating a 4-triazolylphenyl carbamate in good yield and without cleavage of the carbamate linkage.

### SUMMARY OF THE INVENTION

The present invention provides such conditions. Thus, the present invention concerns a process for preparing certain triaryl rhamnose carbamates of the formula (I),

[chemical structure]

wherein

R, R₁ and R₂ independently represent C₁-C₄ alkyl, C₃-C₄ alkenyl or C₁-C₄ fluoroalkyl, and

Z represents a furanyl, phenyl, pyridazinyl, pyridyl, pyrimidinyl or thienyl group, unsubstituted or substituted with one or more substituents independently selected from F, Cl, C₁-C₆ alkyl, C₁-C₆ haloalkyl, C₁-C₆ haloalkoxy or C₁-C₆ haloalkylthio;

which comprises contacting a substituted triazole of formula (II)

[chemical structure]

wherein

Y represents Cl, Br, I, OSO₂CF₃, OSO₂CH₃ or OSO₂C₆H₄CH₃, and

Z is as previously defined,

with a boronic acid or ester of the formula (III)

[chemical structure]

wherein

R, R₁ and R₂ are as previously defined, and

R₃ and R₄ independently represent H, C₁-C₄ alkyl, or when taken together form an ethylene or propylene group optionally substituted with from one to four CH₃ groups, in an ether solvent in the presence of tetrakis(triphenylphosphine)palladium(0) (Pd(PPh₃)₄) and from about 1 to about 2 equivalents of an aqueous alkali metal carbonate at a temperature from about 50° C. to about 100° C.

Another embodiment concerns a boronic acid or ester of the formula (III)

[chemical structure]

wherein

R, R₁ and R₂ independently represent C₁-C₄ alkyl, C₃-C₄ alkenyl or C₁-C₄ fluoroalkyl, and

R₃ and R₄ independently represent H, C₁-C₄ alkyl, or when taken together form an ethylene or propylene group optionally substituted with from one to four CH₃ groups.

In a further embodiment, the boronic ester of the formula (IIIa)

[chemical structure]

wherein

R, R₁ and R₂ independently represent C₁-C₄ alkyl, C₃-C₄ alkenyl or C₁-C₄ fluoroalkyl, and

R₃ₐ and R₄ₐ independently represent C₁-C₄ alkyl, or when taken together form an ethylene or propylene group optionally substituted with from one to four CH₃ groups,

is prepared by a process which comprises

a) contacting p-bromophenyl isocyanate

[chemical structure]

with a tetrahydropyran-2-ol of formula (IV)

[chemical structure]

wherein

R, R₁ and R₂ independently represent C₁-C₄ alkyl, C₃-C₄ alkenyl or C₁-C₄ fluoroalkyl,

in a polar aprotic solvent in the presence of cesium carbonate (Cs₂CO₃) to form a carbamate of formula (V)

[chemical structure]

wherein R, R₁ and R₂ are as previously defined, and

b) contacting the carbamate of formula (V) with a diboron compound of formula (VI)

[chemical structure]

wherein R₃ₐ and R₄ₐ are as previously defined,
in a polar aprotic solvent in the presence of a palladium catalyst and an alkali metal or alkaline earth metal acetate.

In an alternative embodiment, the boronic ester of the formula (IIIa)

[chemical structure]

wherein

R, R₁ and R₂ independently represent C₁-C₄ alkyl, C₃-C₄ alkenyl or C₁-C₄ fluoroalkyl, and

R₃ₐ and R₄ₐ independently represent C₁-C₄ alkyl, or when taken together form an ethylene or propylene group optionally substituted with from one to four CH₃ groups,

is prepared by a process which comprises contacting a boronate substituted phenyl isocyanate of formula (VII)

[chemical structure]

wherein

R₃ₐ and R₄ₐ independently represent C₁-C₄ alkyl, or when taken together form an ethylene or propylene group optionally substituted with from one to four CH₃ groups,

with a tetrahydropyran-2-ol of formula (IV)

[chemical structure]

wherein

R, R₁ and R₂ independently represent C₁-C₄ alkyl, C₃-C₄ alkenyl or C₁-C₄ fluoroalkyl,

in a polar aprotic solvent in the presence of Cs₂CO₃.

Another embodiment concerns a substituted triazole of formula (II)

[chemical structure]

wherein

Y represents Cl, Br, I, OSO₂CF₃, OSO₂CH₃ or OSO₂C₆H₄CH₃, and

Z represents a furanyl, phenyl, pyridazinyl, pyridyl, pyrimidinyl or thienyl group, unsubstituted or substituted with one or more substituents independently selected from F, Cl, C₁-C₆ alkyl, C₁-C₆ haloalkyl, C₁-C₆ haloalkoxy or C₁-C₆ haloalkylthio.

In a further embodiment, the substituted triazole of formula (IIa)

[chemical structure]

wherein
Z represents a furanyl, phenyl, pyridazinyl, pyridyl, pyrimidinyl or thienyl group, unsubstituted or substituted with one or more substituents independently selected from F, Cl, C₁-C₆ alkyl, C₁-C₆ haloalkyl, C₁-C₆ haloalkoxy or C₁-C₆ haloalkylthio,
   is prepared by a process which comprises contacting 3-bromo-1H-1,2,4-triazole

[chemical structure]

with a brominated or iodinated furanyl, phenyl, pyridazinyl, pyridyl, pyrimidinyl or thienyl compound, unsubstituted or substituted with one or more substituents independently selected from F, Cl, C₁-C₆ alkyl, C₁-C₆ haloalkyl, C₁-C₆ haloalkoxy or C₁-C₆ haloalkylthio of one of the following formulas

[chemical structure]

wherein
L represents Br or I,
X independently represents F, Cl, C₁-C₆ alkyl, C₁-C₆ haloalkyl, C₁-C₆ haloalkoxy or C₁-C₆ haloalkylthio,
m=0, 1, 2 or 3,
n=0, 1, 2, 3 or 4, and
p=0, 1, 2, 3, 4 or 5,
   in a polar aprotic solvent in the presence of a catalytic amount of a copper catalyst and at least one equivalent of an inorganic base at a temperature from ambient to about 120° C. The reaction may optionally be conducted in the presence of a complexing ligand for copper.

In an alternative embodiment, the substituted triazole of formula (II)

[chemical structure]

wherein
Y represents Cl, Br, I, OSO₂CF₃, OSO₂CH₃ or OSO₂C₆H₄CH₃, and
Z represents a furanyl, phenyl, pyridazinyl, pyridyl, pyrimidinyl or thienyl group, unsubstituted or substituted with one or more substituents independently selected from F, Cl, C₁-C₆ alkyl, C₁-C₆ haloalkyl, C₁-C₆ haloalkoxy or C₁-C₆ haloalkylthio,
   is prepared by a process which comprises

a) contacting a hydrazine hydrochloride of the formula

Z—NH—NH₂.HCl

wherein
Z represents a furanyl, phenyl, pyridazinyl, pyridyl, pyrimidinyl or thienyl group, unsubstituted or substituted with one or more substituents independently selected from F, Cl, C₁-C₆ alkyl, C₁-C₆ haloalkyl, C₁-C₆ haloalkoxy or C₁-C₆ haloalkylthio,
   with urea in an aprotic organic solvent with a boiling point greater than 100° C. in the presence of a catalytic amount of an organic sulfonic acid at a temperature from about 100° C. to about 150° C.,

b) further contacting the reaction mixture from step a) with a C₁-C₄ alkyl orthoformate and a catalytic amount of chlorosulfonic acid at a temperature from about 60° C. to about 100° C. to provide a substituted 1-H-1,2,4-triazol-3-ol of formula (VIII)

[chemical structure]

wherein Z is as previously defined, and

c) converting the hydroxyl group of the triazole to a Cl, Br, I, OSO₂CF₃, OSO₂CH₃ or OSO₂C₆H₄CH₃.

### DETAILED DESCRIPTION

Throughout this document, all temperatures are given in degrees Celsius, and all percentages are weight percentages unless otherwise stated.

The term “alkyl”, as well as derivative terms such as “haloalkyl”, “fluoroalkyl”, “haloalkoxy” or “haloalkylthio”, as used herein, include within their scope straight chain, branched chain and cyclic moieties. Thus, typical alkyl groups are methyl, ethyl, propyl, butyl, pentyl, hexyl, 1-methylethyl, 1,1-dimethylethyl, 1-methylpropyl, 2-methylpropyl, cyclopropyl, cyclobutyl, cyclopentyl and cyclohexyl. The term “haloalkyl” includes alkyl groups substituted with from one to the maximum possible number of halogen atoms, all combinations of halogens included. The term “haloalkoxy” includes alkoxy groups substituted with from one to the maximum possible number of halogen atoms, all combinations of halogens included. The term “haloalkylthio” includes alkylthio groups substituted with from one to the maximum possible number of halogen atoms, all combinations of halogens included. The term “halogen” or “halo” includes fluorine, chlorine, bromine and iodine.

The furanyl, phenyl, pyridazinyl, pyridyl, pyrimidinyl or thienyl groups may be unsubstituted or substituted with one or more substituents independently selected from F, Cl, C₁-C₆ alkyl, C₁-C₆ haloalkyl, C₁-C₆ haloalkoxy or C₁-C₆ haloalkylthio, provided that the substituents are sterically compatible and the rules of chemical bonding and strain energy are satisfied.

The present invention concerns a process for preparing certain triaryl rhamnose carbamates of the formula (I),

[chemical structure]

wherein

R, R₁ and R₂ independently represent C₁-C₄ alkyl, C₃-C₄ alkenyl or C₁-C₄ fluoroalkyl, and

Z represents a furanyl, phenyl, pyridazinyl, pyridyl, pyrimidinyl or thienyl group, unsubstituted or substituted with one or more substituents independently selected from F, Cl, C₁-C₆ alkyl, C₁-C₆ haloalkyl, C₁-C₆ haloalkoxy or C₁-C₆ haloalkylthio by a Suzuki coupling reaction in good yield under conditions in which the rhamnose carbamate moiety remains intact. This is accomplished by coupling a substituted triazole of formula (II)

[chemical structure]

wherein

Y represents Cl, Br, I, OSO₂CF₃, OSO₂CH₃ or OSO₂C₆H₄CH₃, and

Z is as previously defined

with a boronic acid or ester of the formula (III)

[chemical structure]

wherein

R, R₁ and R₂ independently represent C₁-C₄ alkyl, C₃-C₄ alkenyl or C₁-C₄ fluoroalkyl, and

R₃ and R₄ independently represent H, C₁-C₄ alkyl, or when taken together form an ethylene or propylene group optionally substituted with from one to four CH₃ groups,

in an ether solvent in the presence of tetrakis(triphenylphosphine)palladium(0) (Pd(PPh₃)₄) and from about 1 to about 2 equivalents of an aqueous alkali metal carbonate at a temperature from about 50° C. to about 100° C.

R is preferably CH₃; R₁ is preferably CH₃, CH₂CH₃, CH₂CH₂CH₃ or CH₂CH═CH₂; R₂ is preferably CH₃.

R₃ and R₄ are preferably both CH₃, CH₂CH₃ or CH₂CH₂CH₃ or, when taken together, form an ethylene or propylene group optionally substituted with from one to four CH₃ groups.

Z is preferably a phenyl group substituted with a C₁-C₆ haloalkoxy group, most preferably with a C₁-C₂ fluoroalkoxy group in the para position.

Y is preferably Br.

The coupling reaction is conducted in an ether solvent. Preferred solvents are miscible with water and include THF, dioxane and dimethoxyethane (DME), with DME being most preferred.

The coupling reaction is run in the presence of Pd(PPh₃)₄. From about 0.05 to about 0.10 molar equivalents of this material is preferred, but, with particularly unreactive substrates, up to almost a stoichiometric amount may be needed.

The coupling reaction requires at least one equivalent of an aqueous alkali metal carbonate base, but a 2- to 3-fold excess of base is often recommended. To preserve the integrity of the carbamates-rhamnose moiety, it is important to use from about 1 to about 2 equivalents of an aqueous alkali metal carbonate. The preferred alkali metal carbonate is sodium carbonate (Na₂CO₃).

The coupling reaction is conducted at a temperature from about 50° C. to about 100° C., with a temperature from about 70° C. to about 90° C. being preferred.

In a typical reaction, the substituted 3-bromotriazole, the boronic ester of the carbamate-rhamnose, 1 equivalent of aqueous Na₂CO₃ and 10 mol % Pd(PPh₃)₄ are sealed in a vessel with DME. The reaction is heated at 90° C. until the reaction is completed. The reaction mixture is cooled, diluted with a water insoluble organic solvent and water and the organic phase partitioned. The solvent is evaporated and the isolated product purified by conventional techniques such as preparative reverse phase chromatography.

The starting boronic esters of the formula (Ma)

[chemical structure]

wherein

R, R₁ and R₂ independently represent C₁-C₄ alkyl, C₃-C₄ alkenyl or C₁-C₄ fluoroalkyl, and

R₃ₐ and R₄ₐ independently represent C₁-C₄ alkyl, or when taken together form an ethylene or propylene group optionally substituted with from one to four CH₃ groups are novel materials and are prepared by two different approaches.

The first process comprises

a) contacting p-bromophenyl isocyanate

[chemical structure]

with a tetrahydropyran-2-ol of formula (IV)

[chemical structure]

wherein

R, R₁ and R₂ independently represent C₁-C₄ alkyl, C₃-C₄ alkenyl or C₁-C₄ fluoroalkyl,

in a polar aprotic solvent in the presence of Cs₂CO₃ to form a (4-bromophenyl)carbamate of formula (V)

[chemical structure]

wherein R, R₁ and R₂ are as previously defined, and

b) contacting the carbamate of formula (V) with a diboron compound of formula (VI)

[chemical structure]

wherein R₃ₐ and R₄ₐ are as previously defined,

in a polar aprotic solvent in the presence of a palladium catalyst and an alkali metal or alkaline earth metal acetate.

In the first step, the p-bromophenyl isocyanate is contacted with the tetrahydropyran-2-ol in a polar aprotic solvent which includes amides, like N, N-dimethylformamide (DMF), N, N-dimethylacetamide (DMA) or N-methyl-2-pyrrolidinone (NMP), sulfoxides, like dimethyl sulfoxide (DMSO), esters, like ethyl acetate (EtOAc), and nitriles, like acetonitrile (MeCN), butyronitrile or benzonitrile. Nitriles, particularly MeCN, are preferred. The polar aprotic solvent should be as anhydrous as possible to avoid hydrolysis of the isocyanate and the formation of byproduct ureas.

The first step is run in the presence of Cs₂CO₃, usually in the presence of from about 1 to about 2 equivalents.

The first step is conducted at a temperature from about 0° C. to about 90° C., with a temperature from about 0° C. to about 35° C. being preferred. The tetrahydropyran-2-ol (IV) normally exists as a mixture of anomeric forms, cc and 13. During the course of the reaction to form the carbamate, both the cc and 13 anomers are initially formed. With continued stirring after the isocyanate has been converted entirely into the mixture of carbamates, further equilibration occurs, resulting ultimately in exclusive formation of the cc anomer.

In a typical reaction, the p-bromophenyl isocyanate and Cs₂CO₃, are added to the tetrahydropyran-2-ol in MeCN. The reaction is stirred at room temperature until the reaction and equilibration are completed. The reaction mixture is filtered to remove solids, the solvent is evaporated and the isolated product purified by conventional techniques such as flash chromatography.

In the second step, the (4-bromophenyl)carbamate is contacted with a diboron compound of formula VI

[chemical structure]

wherein R₃ₐ and R₄ₐ are as previously defined,

in a polar aprotic solvent in the presence of a palladium catalyst and an alkali metal or alkaline earth metal acetate.

The second step is also run in a polar aprotic solvent, which likewise includes amides, like DMF, DMA or NMP, sulfoxides, like DMSO, esters, like EtOAc, and nitriles, like MeCN, butyronitrile and benzonitrile. While it is possible to run the second step using the reaction mixture of the first step without isolation and purification of the (4-bromophenyl)carbamates, and thus use the same solvent as employed in the first step, it is preferable to use a sulfoxide solvent such as DMSO.

The second step is run in the presence of a catalytic amount of palladium catalyst. A catalytic amount means from about 0.01 to about 0.20 equivalents of a palladium catalyst. From about 0.05 to about 0.10 equivalents of catalyst is preferred. The palladium catalyst may be Pd(0), such as Pd(PPh₃)₄, or Pd(II) such as [1,1′-bis(diphenylphosphino)ferrocene]dichloropalladium(II) (PdCl₂(dppf)) or bis(diphenylphosphino)dichloropalladium(II) (PdCl₂(PPh₃)₂).

The second step requires at least one equivalent of an alkali metal or alkaline earth metal acetate, but a large excess is often recommended. It is generally preferred to use from about 1.5 to about 3 equivalents of alkali metal or alkaline earth metal acetate. The preferred alkali metal or alkaline earth metal acetate is sodium (NaOAc) or potassium acetate (KOAc).

The second step is conducted at a temperature from about 50° C. to about 110° C., with a temperature from about 70° C. to about 90° C. being preferred.

In a typical reaction, the p-bromophenyl carbamate, the diboron compound, the palladium catalyst and the alkali metal or alkaline earth metal acetate are charged into a reaction vessel. The reaction vessel is sealed and is evacuated and backfilled with nitrogen (N₂) multiple times. The polar aprotic solvent is added and the mixture heated at about 80° C. until the reaction is completed. The reaction mixture is cooled, diluted with water and extracted with ether. The ether extract is dried and evaporated and the isolated product purified by conventional techniques such as flash chromatography.

Alternatively, the second process comprises contacting a boronate substituted phenyl isocyanate of formula (VII)

[chemical structure]

wherein

R₃ₐ and R₄ₐ independently represent C₁-C₄ alkyl, or when taken together form an ethylene or propylene group optionally substituted with from one to four CH₃ groups,

with a tetrahydropyran-2-ol of formula (IV)

[chemical structure]

wherein

R, R₁ and R₂ independently represent C₁-C₄ alkyl, C₃-C₄ alkenyl or C₁-C₄ fluoroalkyl, in a polar aprotic solvent in the presence of Cs₂CO₃.

In the second reaction, the boronate substituted phenyl isocyanate is contacted with the tetrahydropyran-2-ol in a polar aprotic solvent which includes amides, like DMF, DMA or NMP, sulfoxides, like DMSO, esters, like EtOAc, and nitriles, like MeCN, butyronitrile and benzonitrile. Nitriles, particularly MeCN, are preferred. The polar aprotic solvent should be as anhydrous as possible to avoid hydrolysis of the isocyanate and the formation of byproduct ureas.

The second reaction is run in the presence of Cs₂CO₃, usually in the presence of from about 1 to about 2 equivalents.

The second reaction is conducted at a temperature from about 0° C. to about 90° C., with a temperature from about 0° C. to about 35° C. being preferred. The tetrahydropyran-2-ol (IV) normally exists as a mixture of anomeric forms, cc and 13. During the course of the reaction to form the carbamate, both the α and β anomers are initially formed. With continued stirring after the isocyanate has been converted entirely into the mixture of carbamates, further equilibration occurs, resulting ultimately in exclusive formation of the cc anomer.

In a typical reaction, the boronate substituted phenyl isocyanate and Cs₂CO₃, are added to the tetrahydropyran-2-ol in MeCN. The reaction is stirred at room temperature until the reaction and equilibration are completed. The reaction mixture is filtered to remove solids, the solvent is evaporated and the isolated product purified by conventional techniques such as flash chromatography.

The starting substituted triazoles of formula (II)

[chemical structure]

wherein

Y represents Cl, Br, I, OSO₂CF₃, OSO₂CH₃ or OSO₂C₆H₄CH₃, and

Z represents a furanyl, phenyl, pyridazinyl, pyridyl, pyrimidinyl or thienyl group, unsubstituted or substituted with one or more substituents independently selected from F, Cl, C₁-C₆ alkyl, C₁-C₆ haloalkyl, C₁-C₆ haloalkoxy or C₁-C₆ haloalkylthio

are novel materials and are prepared by two different approaches.

The first process comprises contacting 3-bromo-1H-1,2,4-triazole

[chemical structure]

with a brominated or iodinated furanyl, phenyl, pyridazinyl, pyridyl, pyrimidinyl or thienyl compound, unsubstituted or substituted with one or more substituents independently selected from F, Cl, C₁-C₆ alkyl, C₁-C₆ haloalkyl, C₁-C₆ haloalkoxy or C₁-C₆ haloalkylthio of one of the following formulas

[chemical structure]

wherein
L represents Br or I,
X independently represents F, Cl, C₁-C₆ alkyl, C₁-C₆ haloalkyl, C₁-C₆ haloalkoxy or C₁-C₆ haloalkylthio,
m=0, 1, 2 or 3,
n=0, 1, 2, 3 or 4, and
p=0, 1, 2, 3, 4 or 5,
   in a polar aprotic solvent in the presence of a catalytic amount of a copper catalyst and at least one equivalent of an inorganic base at a temperature from ambient to about 120° C. The reaction is usually conducted at a temperature from about 80° C. to about 120° C. The reaction may optionally be conducted in the presence of a complexing ligand for copper. In the case of more activated haloheterocycles, such as 3-chloro-2-fluoro-5-(trifluoromethyl)pyridine, this coupling could be run at room temperature without the need for a copper catalyst.

In the first process, the 3-bromo-1H-1,2,4-triazole is contacted with the brominated or iodinated furanyl, phenyl, pyridazinyl, pyridyl, pyrimidinyl or thienyl compound in a polar aprotic solvent which includes amides, like DMF, DMA or NMP and sulfoxides, like DMSO. DMSO is particularly preferred. The polar aprotic solvent should be as anhydrous as possible.

The first process is run in the presence of catalytic amount of copper catalyst, usually in the presence of from about 0.05 to about 0.25 equivalents. About 0.1 to about 0.2 equivalents of copper catalyst is preferred. Cuprous salts are generally preferred as the copper catalyst, with cuprous iodide (CuI) being especially preferred.

The first process is also run in the presence of at least one equivalent of an inorganic base, usually in the presence of from about 1 to about 2 equivalents. Preferred inorganic bases are the alkali metal carbonates and phosphates such as sodium, potassium and cesium carbonates and phosphates, with Cs₂CO₃ being particularly preferred.

The first process may optionally be conducted in the presence of an amine-containing ligand which complexes with the copper reagent such as cyclohexyl diamine or dimethylethane-1,2-diamine. However, rather than including such an additional material, it has been found that performing the first process with an excess of the 3-bromo-1H-1,2,4-triazole is beneficial. From about 1.5 to about 3.0 equivalents of 3-bromo-1H-1,2,4-triazole per equivalent of brominated or iodinated furanyl, phenyl, pyridazinyl, pyridyl, pyrimidinyl or thienyl compound is preferred.

The first process is conducted at a temperature from ambient to about 120° C., with a temperature from about 80° C. to about 120° C. being preferred.

In a typical reaction, the inorganic base, CuI and the brominated triazole are charged to a reaction vessel which is evacuated and backfilled with N₂ three times. The polar aprotic solvent, brominated or iodinated furanyl, phenyl, pyridazinyl, pyridyl, pyrimidinyl or thienyl compound and any complexing ligand are added and the mixture is heated at a temperature from about 80° C. to about 120° C. until the reaction is complete. The reaction mixture is cooled, diluted with a water immiscible organic solvent and filtered to remove solids. The organic filtrate is washed with a dilute aqueous acid and dried and the solvent is evaporated and the isolated product purified by conventional techniques such as flash chromatography.

Alternatively, the second process comprises the preparation of a substituted triazole of formula (II)

[chemical structure]

wherein
Y represents Cl, Br, I, OSO₂CF₃, OSO₂CH₃ or OSO₂C₆H₄CH₃, and
Z represents a furanyl, phenyl, pyridazinyl, pyridyl, pyrimidinyl or thienyl group, unsubstituted or substituted with one or more substituents independently selected from F, Cl, C₁-C₆ alkyl, C₁-C₆ haloalkyl, C₁-C₆ haloalkoxy or C₁-C₆ haloalkylthio,
   by

a) contacting a hydrazine hydrochloride of the formula

Z—NH—NH₂.HCl

wherein
Z represents a furanyl, phenyl, pyridazinyl, pyridyl, pyrimidinyl or thienyl group, unsubstituted or substituted with one or more substituents independently selected from F, Cl, C₁-C₆ alkyl, C₁-C₆ haloalkyl, C₁-C₆ haloalkoxy or C₁-C₆ haloalkylthio,
   with urea in an aprotic organic solvent with a boiling point greater than 100° C. in the presence of a catalytic amount of an organic sulfonic acid at a temperature from about 100° C. to about 150° C.,

b) further contacting the reaction mixture from step a) with a C₁-C₄ alkyl orthoformate and a catalytic amount of chlorosulfonic acid at a temperature from about 60° C. to about 100° C. to provide a substituted 1-H-1,2,4-triazol-3-ol of formula (VIII)

[chemical structure]

wherein Z is as previously defined, and

c) converting the hydroxyl group of the triazole to a Cl, Br, I, OSO₂CF₃, OSO₂CH₃ or OSO₂C₆H₄CH₃.

In the initial step of the second process, the substituted hydrazine hydrochloride is contacted with urea in an aprotic organic solvent with a boiling point greater than 100° C.

The substituted hydrazines are conveniently prepared from the corresponding amino compounds by reaction with sodium nitrite (NaNO₂) to produce a diazonium salt, followed by reduction with a reducing agent such as hydrogen, sodium dithionite (Na₂S₂O₄), tin (II) chloride (SnCl₂) or ammonium formate to provide the hydrazine. It is beneficial to employ up to a 50 mol % excess of urea. Most suitable aprotic organic solvents include inert hydrocarbons and halogenated hydrocarbons. Chlorobenzene is particularly preferred.

The initial step of the second process is run in the presence of catalytic amount of an organic sulfonic acid, usually in the presence of from about 0.05 to about 0.25 equivalents. About 0.1 to about 0.2 equivalents of the organic sulfonic acid is preferred.

The initial step of the second process is conducted at a temperature from about 100° C. to about 150° C., with a temperature from about 110° C. to about 140° C. being preferred.

In the second step of the second process the reaction mixture from the initial step is further contacted with a C₁-C₄ alkyl orthoformate and a catalytic amount of chlorosulfonic acid at a temperature from about 60° C. to about 100° C. to provide a substituted 1-H-1,2,4-triazol-3-ol.

The second step of the second process is run with at least one equivalent of orthoformate; usually a slight excess of about 0.1 to about 0.2 equivalents of the orthoformate is preferred.

The second step of the second process is run in the presence of catalytic amount of chlorosulfonic acid, usually in the presence of from about 0.01 to about 0.2 equivalents. About 0.01 to about 0.1 equivalents of the chlorosulfonic acid is preferred.

The second step of the second process is conducted at a temperature from about 60° C. to about 100° C., with a temperature from about 70° C. to about 90° C. being preferred.

In a typical process, the first two reaction steps are performed sequentially without isolation. The substituted hydrazine hydrochloride, urea and organic sulfonic acid are suspended in an aprotic organic solvent with a boiling point greater than 100° C. and refluxed until the reaction is complete. The mixture is cooled to about 80° C. and treated with the orthoformate and chlorosulfonic acid. After completion of the reaction, the mixture is then cooled to room temperature and filtered. The solvent is evaporated and the residue dried under vacuum.

In the third step of the second process the hydroxyl group is converted to a Cl, Br, I, OSO₂CF₃, OSO₂CH₃ or OSO₂C₆H₄CH₃ group by procedures well known to those of ordinary skill in the art. For example, Cl, Br, and I groups are introduced by halo de-hydoxylation reactions using halogenated Brønsted acids such as hydrochloric acid (HCl), hydrobromic acid (HBr) and hydroiodic acid (HI) or halogenated Lewis acids such as phosphorus trichloride (PCl₃), phosphoryl chloride (POCl₃), sulfonyl chloride (SOCl₂) or phosphoryl bromide (POBr₃). The OSO₂CF₃, OSO₂CH₃

or OSO₂C₆H₄CH₃ groups are introduced by esterification of sulfonic acid anhydrides or halides.

The following examples are presented to illustrate the invention.

### Examples

### Example 1

### Preparation of (2R,3S,4S,5R,6R)-4-ethoxy-3,5-dimethoxy-6-methyltetrahydro-2H-pyran-2-yl{{4-{1-[4-(trifluoromethoxy)phenyl]-1H-1,2,4-triazol-3-yl}phenyl}}carbamate

[chemical structure]

A microwave vial was charged with (2R,3S,4S,5R,6R)-4-ethoxy-3,5-dimethoxy-6-methyltetrahydro-2H-pyran-2-yl(4-(4,4,5,5-tetramethyl-1,3,2-dioxaborolan-2-yl)phenyl)carbamate (200 mg, 0.430 mmol), 3-bromo-1-(4-(trifluoromethoxy)phenyl)-1H-1,2,4-triazole (159 mg, 0.516 mmol), aqueous Na₂CO₃ (1 M, 0.8 mL), and Pd(PPh₃)₄ (49.7 mg, 0.043 mmol). The reaction vial was sealed, DME (4.3 mL) was added, and the reaction was heated at 90° C. for 6 hours (h) in a Biotage Initiator® microwave reactor with external IR-sensor temperature monitoring from the side of the vessel. The reaction mixture was cooled to room temperature, diluted with dichloromethane (CH₂Cl₂), and water was added. The layers were separated with a phase separator and the organics were concentrated in vacuo. Purification via reverse phase chromatography yielded the title compound as a white solid (184 mg, 73%): ¹H NMR (400 MHz, CDCl₃) δ 8.55 (s, 1H), 8.16 (m, 1H), 7.79 (m, 2H), 7.53 (m, 1H), 7.40 (m, 3H), 6.75 (d, J=30.8 Hz, 1H), 6.19 (dd, J=9.5, 1.9 Hz, 1H), 3.69 (m, 4H), 3.60 (m, 4H), 3.55 (s, 1H), 3.21 (td, J=9.4, 6.0 Hz, 1H), 1.32 (m, 9H); ¹⁹F NMR (376 MHz, CDCl₃) δ −58.03; ESIMS m/z 567.2 ([M+H]⁺).

### Example 2

### Preparation of (2S,3R,4R,5S,6S)-4-ethoxy-3,5-dimethoxy-6-methyltetrahydro-2H-pyran-2-yl(4-bromophenyl)carbamate

[chemical structure]

To (3R,4R,5S,6S)-4-ethoxy-3,5-dimethoxy-6-methyltetrahydro-2H-pyran-2-ol (311.1 mg, 1.412 mmol) in MeCN (10 mL) was added p-bromophenyl isocyanate (282.9 mg, 1.429 mmol) followed by Cs₂CO₃ (502.5 mg, 1.542 mmol). The reaction mixture was allowed to stir at room temperature until consumption of the starting material was complete. Upon completion of the reaction, the mixture was filtered to remove solids. The filtrate was concentrated in vacuo. Purification via flash column chromatography using 100% CH₂Cl₂ to 10% MeCN/CH₂Cl₂ (v/v) as eluent yielded the title compound as a white solid (400 mg, 66.4%): ¹H NMR (400 MHz, CDCl₃) δ 7.43 (m, 2H), 7.31 (d, J=8.3 Hz, 2H), 6.68 (s, 1H), 6.16 (d, J=1.9 Hz, 1H), 3.67 (m, 3H), 3.59 (s, 4H), 3.55 (s, 4H), 3.20 (t, J=9.4 Hz, 1H), 1.30 (m, 6H).

### Example 3

### Preparation of (2R,3S,4S,5R,6R)-4-ethoxy-3,5-dimethoxy-6-methyltetrahydro-2H-pyran-2-yl(4-(4,4,5,5-tetramethyl-1,3,2-dioxaborolan-2-yl)phenyl)carbamate

[chemical structure]

To a dry flask was added (2R,3S,4S,5R,6R)-4-ethoxy-3,5-dimethoxy-6-methyltetrahydro-2H-pyran-2-yl(4-bromophenyl)carbamate (0.2 g, 0.478 mmol), PdCl₂(dppf) (0.04 g, 0.048 mmol), bis(pinacolato)diboron (0.127 g, 0.502 mmol), and KOAc (0.141 g, 1.43 mmol). The vial was sealed, and evacuated/backfilled with N₂ (3×). DMSO (1.594 mL) was added, and the reaction mixture was heated to 70° C. until consumption of the starting material was complete as verified by UPLC analysis (˜6 h). The reaction was cooled to room temperature, diluted with water and extracted with diethyl ether. The aqueous phase was further extracted with diethyl ether (2×). The organics were combined, dried and concentrated in vacuo. Purification via flash column chromatography (EtOAc/hexanes) afforded the title compound as a white foam (120 mg, 52.9%): ¹H NMR (400 MHz, CDCl₃) δ 7.77 (m, 2H), 7.41 (d, J=8.0 Hz, 2H), 6.70 (s, 1H), 6.18 (d, J=1.9 Hz, 1H), 3.74 (dd, J=9.3, 7.0 Hz, 1H), 3.65 (m, 3H), 3.59 (s, 3H), 3.55 (s, 4H), 3.20 (t, J=9.4 Hz, 1H), 1.33 (d, J=5.9 Hz, 13H), 1.29 (m, 5H); ESIMS m/z 464.4 ([M−H]⁻); IR (thin film) 3311, 2978, 1733 cm⁻¹.

### Example 4

### Preparation of (2S,3R,4R,5S,6S)-4-ethoxy-3,5-dimethoxy-6-methyltetrahydro-2H-pyran-2-yl(4-(4,4,5,5-tetramethyl-1,3,2-dioxaborolan-2-yl)phenyl)carbamate

[chemical structure]

To (3R,4R,5S,6S)-4-ethoxy-3,5-dimethoxy-6-methyltetrahydro-2H-pyran-2-ol (3.0598 g, 13.89 mmol) in MeCN (150 mL) at 0° C. was added 2-(4-isocyanatophenyl)-4,4,5,5-tetramethyl-1,3,2-dioxaborolane (5.000 g, 20.40 mmol) followed by Cs₂CO₃ (4.643 g, 14.25 mmol). The mixture was stirred at 0° C. for 10 minutes (min) and was then allowed to warm to room temperature and stir until consumption of the starting material was complete (˜1 h). The reaction mixture was filtered through Celite®, rinsing with fresh MeCN. The filtrates were combined and concentrated in vacuo. Purification via flash column chromatography (EtOAc/hexanes) afforded the title compound as a colorless solid (4.3105 g, 67%, a isomer only): ¹H NMR (400 MHz, CDCl₃) δ 7.77 (m, 2H), 7.42 (m, 2H), 6.78 (s, 1H), 6.18 (d, J=1.9 Hz, 1H), 3.67 (m, 4H), 3.59 (s, 3H), 3.55 (s, 3H), 3.20 (t, J=9.4 Hz, 1H), 1.34 (s, 12H), 1.28 (m, 7H); ¹³C NMR (101 MHz, CDCl₃) δ 171.21, 151.03, 139.89, 135.94, 117.59, 92.00, 83.77, 83.68, 81.45, 79.28, 70.40, 65.81, 61.17, 60.41, 59.21, 24.87, 21.06, 17.90, 15.71, 14.20; ESIMS m/z 466.3 ([M+H]⁺).

### Example 5

### Preparation of 3-bromo-1-(4-(trifluoromethoxy)phenyl)-1H-1,2,4-triazole

[chemical structure]

A dry round bottom flask was charged with potassium phosphate (K₃PO₄, 7.74 g, 36.5 mmol), CuI (0.165 g, 0.868 mmol), and 3-bromo-1H-1,2,4-triazole (2.83 g, 19.10 mmol). The flask was evacuated/backfilled with N₂ (3×). DMF (34.7 mL) was added, followed by trans-(1R,2R)—N,N′-bismethyl-1,2-cyclohexane diamine (0.274 ml, 1.736 mmol) and 1-iodo-4-(trifluoromethoxy)benzene (5.000 g, 17.36 mmol). The solution was heated to 110° C. After 48 h, the reaction mixture was cooled to room temperature, diluted with EtOAc and filtered through Celite®. The filtrate was washed with water (100 mL) containing HCl (1 M, 10 mL. The organics were separated, and the aqueous phase was further extracted with EtOAc (3×). The organics were combined, dried, and concentrated in vacuo. Purification via flash column chromatography (EtOAc/hexanes) yielded the title compound as a tan solid (1.86 g, 34%): ¹H NMR (400 MHz, CDCl₃) δ 8.44 (s, 1H), 7.70 (d, J=8.9 Hz, 2H), 7.38 (d, J=8.5 Hz, 2H); ¹⁹F NMR (376 MHz, CDCl₃) δ −58.04; EIMS m/z 307.

### Example 6

### Preparation of 3-bromo-1-(4-(trifluoromethoxy)phenyl)-1H-1,2,4-triazole

[chemical structure]

A dry round bottom flask was charged with 3-bromo-1H-1,2,4-triazole (5 g, 33.8 mmol), CuI (0.644 g, 3.38 mmol), and Cs₂CO₃ (11.01 g, 33.8 mmol). The flask was evacuated/backfilled with N₂, then DMSO (33.8 mL) and 1-iodo-4-(trifluoromethoxy)benzene (4.87 g, 16.90 mmol) were added. The reaction mixture was heated to 100° C. for 36 h. The reaction mixture was cooled to room temperature, diluted with EtOAc, filtered through a plug of Celite® and further washed with EtOAc. Water was added to the combined organics, and the layers were separated. The aqueous phase was neutralized to pH 7, and further extracted with EtOAc. The combined organics were concentrated in vacuo. Purification via flash column chromatography (EtOAc/hexanes) yielded the title compound as an off white solid (3.78 g, 72.6%): mp 67-69° C.; ¹H NMR (400 MHz, CDCl₃) δ 8.43 (s, 1H), 7.70 (m, 2H), 7.38 (m, 2H); ¹⁹F NMR (376 MHz, CDCl₃) δ −58.02.

### Example 7

### Preparation of (4-(perfluoroethoxy)phenyl)hydrazine

[chemical structure]

To a dry 500 mL round bottom flask equipped with magnetic stirrer, N₂ inlet, addition funnel, and thermometer, were charged 4-perfluoroethoxyaniline (11.8 g, 52.0 mmol) and HCl (2 N, 100 mL), and the resulting suspension was cooled to about 0° C. with an external ice/salt (sodium chloride, NaCl) bath. To the suspension was added a solution of NaNO₂ (1.05 g, 54.5 mmol) in water; (10 mL) dropwise from the addition funnel at a rate which maintained the temperature below 5° C., and the resulting colorless solution was stirred at 0° C. for 30 min. To a separate 500 mL round bottomed flask equipped with magnetic stir bar, addition funnel, and thermometer were added Na₂S₂O₄ (27.1 g, 156 mmol), sodium hydroxide (NaOH; 1.04 g, 26.0 mmol), and water (60 mL), and the suspension was cooled to about 5° C. with an external cooling bath. The diazonium salt solution prepared in round bottom 1 was transferred to the addition funnel and added to round bottom 2 containing the aqueous Na₂S₂O₄/NaOH suspension at a rate which maintained the temperature below 8° C. Following the addition, the reaction mixture was warmed to 18° C. and the pH was adjusted to about 8 with 50% NaOH. The resulting pale orange solution was extracted with EtOAc (3×100 mL) and the combined organic extracts were washed with water (100 mL), washed with saturated aqueous NaCl solution (brine; 100 mL), dried over anhydrous magnesium sulfate (MgSO₄), filtered, and the filtrate concentrated to give the crude product as an orange semi-solid (12.2 g). The residue was purified by flash column chromatography using 0-100% EtOAc/hexanes as eluent to give the title compound as a yellow liquid (10.4 g, 83%): ¹H NMR (400 MHz, CDCl₃) δ 7.18-7.00 (m, 2H), 6.97-6.68 (m, 2H), 5.24 (bs, 1H), 3.98-3.09 (bs, 2H); ¹⁹F NMR (376 MHz, CDCl₃) δ −86.00, −86.01, −87.92; EIMS m/z 242 ([M⁺]).

### Example 8

### Preparation of 1-(4-(Perfluoroethoxy)phenyl)-1H-1,2,4-triazol-3-ol

[chemical structure]

A mixture of (4-(perfluoroethoxy)phenyl)hydrazine hydrochloride (5 g, 17.95 mmol), urea (1.46 g; 24.23 mmol) and p-toluenesulfonic acid (pTSoH, 24 mg, 0.18 mmol) suspended in chlorobenzene (16.3 mL) was refluxed for 2 h (140° C.). The mixture was then cooled to 80° C. and triethyl orthoformate was added (3.20 mL, 19.20 mmol) followed by chlorosulfonic acid (24 μL, 0.36 mmol). The reaction was heated at 80° C. for 4 h. The reaction was cooled to room temperature and filtered. The residue was dried under high vacuum overnight to give the title compound as a white solid (5.24 g, 99%): mp>300° C.; ¹H NMR (400 MHz, DMSO-d₆) δ 11.55 (s, 1H), 8.96 (s, 1H), 7.88 (d, J=9.1 Hz, 2H), 7.54 (d, J=9.1 Hz, 2H); ¹⁹F NMR (376 MHz, DMSO-d₆) δ −85.23, −86.96; ¹³C NMR (101 MHz, DMSO-d₆) δ 167.77, 145.31, 141.44, 135.97, 123.00, 119.85; ESIMS m/z 295 [(M+H)]⁺.

### Example 9

### Preparation of 3-Bromo-1-(4-(Perfluoroethoxy)phenyl)-1H-1,2,4-triazole

[chemical structure]

A suspension containing 1-(4-(perfluoroethoxy)phenyl)-1H-1,2,4-triazol-3-ol (100 mg, 0.34 mmol) and POBr₃ (194 mg, 0.68 mmol) was heated at 170° C. for 2 h. The reaction was cooled to room temperature and quenched by the slow addition of ice. The suspension was extracted with chloroform (CHCl₃). The combined organic layers were dried over MgSO₄, filtered and concentrated. This material was run down a plug of silica gel using CHCl₃ as the eluent to give the title compound (15 mg, 12%): ¹H NMR (400 MHz, CDCl₃) δ 8.43 (s, 1H), 7.81-7.62 (m, 2H), 7.48-7.31 (m, 2H); ¹⁹F NMR (376 MHz, CDCl₃) δ −86.05 (d, J=7.1 Hz), −87.99 (d, J=3.7 Hz); GCMS m/z 358 [(M+2)]^{±}.

### Example 10

### Preparation of 1-(4-(Perfluoroethoxy)phenyl)-1H-1,2,4-triazol-3-yl-trifluoromethane sulfonate

[chemical structure]

To an ice cold solution containing 1-(4-(perfluoroethoxy)phenyl)-1H-1,2,4-triazol-3-ol (558 mg, 1.89 mmol) and triethylamine (TEA, 0.40 mL, 2.84 mmol) dissolved in CH₂Cl₂ (7 mL) was added a solution of trifluoromethane-sulfonic anhydride (0.34 mL, 1.99 mmol) dissolved in CH₂Cl₂ (3 mL) dropwise. The reaction was stirred at 0° C. for 1 h and warmed to room temperature. The mixture was diluted with CH₂Cl₂ and washed with cold water. The solution was dried over MgSO₄, filtered and concentrated. The residue was dissolved in CH₂Cl₂ (10 mL) and added to an loading cartridge containing Celite® and purified via flash column chromatography using EtOAc/hexanes as eluent. The title compound was obtained as a yellow oil (406 mg, 50%): ¹H NMR (400 MHz, CDCl₃) δ 8.43 (s, 1H), 7.72 (d, J=9.2 Hz, 2H), 7.42 (d, J=9.2 Hz, 3H); ¹⁹F NMR (376 MHz, CDCl₃) δ −72.17, −85.90, −87.94; GC/MS m/z 427 [(M+H)]⁺.

### Example 11

### Preparation of (2S,3R,4R,5S,6S)-4-ethoxy-3,5-dimethoxy-6-methyltetrahydro-2H-pyran-2-yl(4-(1-(4-(perfluoroethoxy)phenyl)-1H-1,2,4-triazol-3-yl)phenyl)carbamate

[chemical structure]

To a solution containing 1-(4-(perfluoroethoxy)phenyl)-1H-1,2,4-triazol-3-yl trifluoromethanesulfonate (75 mg, 0.176 mmol) and (2S,3R,4R,5S,6S)-4-ethoxy-3,5-dimethoxy-6-methyl-tetrahydro-2H-pyran-2-yl(4-(4,4,5,5-tetra-methyl-1,3,2-dioxaborolan-2-yl)phenyl)carbamate (82 mg, 0.176 mmol) in DME (1.8 mL) was added aqueous Na₂CO₃ (2M; 0.27 mL; 0.527 mmol). The mixture was degassed by bubbling N₂ through the solution for 5 min. Pd(PPh₃)₄ (41 mg, 0.035 mmol) was then added and the mixture was heated at 85° C. overnight. The mixture was diluted with EtOAc and washed with a saturated solution of sodium bicarbonate (NaHCO₃). The organic phase was dried (MgSO₄), filtered and concentrated. The residue was purified via radial chromatography on silica gel using a 2:1 hexane/EtOAc mixture as the eluent (R_{f}=0.25) to give the title compound (16 mg, 15%): ¹H NMR (400 MHz, CDCl₃) δ 8.56 (s, 1H), 8.17 (d, J=8.8 Hz, 2H), 7.81 (d, J=9.1 Hz, 2H), 7.54 (d, J=8.4 Hz, 2H), 7.40 (d, J=9.0 Hz, 2H), 6.79 (s, 1H), 6.20 (s, 1H), 3.60 (s, 3H) 3.57 (s, 3H), 3.81-3.56 (m, 5H) 3.21 (t, J=9.4 Hz, 1H), 1.45-1.21 (m, 6H); ESIMS m/z 616 [(M+H)]⁺.

What is claimed is:

1. A process for preparing triaryl rhamnose carbamates of the formula (I), [chemical structure]
wherein
R, R₁ and R₂ independently represent C₁-C₄ alkyl, C₃-C₄ alkenyl or C₁-C₄ fluoroalkyl, and
Z represents a furanyl, phenyl, pyridazinyl, pyridyl, pyrimidinyl or thienyl group, unsubstituted or substituted with one or more substituents independently selected from F, Cl, C₁-C₆ alkyl, C₁-C₆ haloalkyl, C₁-C₆ haloalkoxy or C₁-C₆ haloalkylthio;
which comprises contacting a substituted triazole of formula (II) [chemical structure]
wherein
Y represents Cl, Br, I, OSO₂CF₃, OSO₂CH₃ or OSO₂C₆H₄CH₃, and
Z is as previously defined
with a boronic acid or ester of the formula (III) [chemical structure]
wherein
R, R₁ and R₂ are as previously defined, and
R₃ and R₄ independently represent H, C₁-C₄ alkyl, or when taken together form an ethylene or propylene group optionally substituted with from one to four CH₃ groups,
in an ether solvent in the presence of tetrakis(triphenylphosphine)palladium(0) and from about 1 to about 2 equivalents of an aqueous alkali metal carbonate at a temperature from about 50° C. to about 100° C.

2. The process of claim 1 in which R is CH₃; R₁ is CH₃, CH₂CH₃, CH₂CH₂CH₃ or CH₂CH═CH₂; R₂ is CH₃; R₃ and R₄ are both CH₃, CH₂CH₃ or CH₂CH₂CH₃ or, when taken together, form an ethylene or propylene group optionally substituted with from one to four CH₃ groups; Z is a phenyl group substituted with a C₁-C₆ haloalkoxy group; and Y is Br.

3. The process of claim 1 in which about 0.05 to about 0.10 equivalents tetrakis(triphenylphosphine)palladium(0) is used.

4. The process of claim 1 in which the ether solvent is miscible with water.

5. The process of claim 4 in which the ether solvent is tetrahydrofuran, dioxane or dimethoxyethane.

6. A boronic acid or ester of the formula (III) [chemical structure]
wherein
R, R₁ and R₂ independently represent C₁-C₄ alkyl, C₃-C₄ alkenyl or C₁-C₄ fluoroalkyl, and
R₃ and R₄ independently represent H, C₁-C₄ alkyl, or when taken together form an ethylene or propylene group optionally substituted with from one to four CH₃ groups.

7. A process for preparing a boronic ester of the formula (IIIa) [chemical structure]
wherein
R, R₁ and R₂ independently represent C₁-C₄ alkyl, C₃-C₄ alkenyl or C₁-C₄ fluoroalkyl, and
R₃ₐ and R₄ₐ independently represent C₁-C₄ alkyl, or when taken together form an ethylene or propylene group optionally substituted with from one to four CH₃ groups, which comprises
a) contacting p-bromophenyl isocyanate [chemical structure]
with a tetrahydropyran-2-ol of formula (IV) [chemical structure]
wherein
R, R₁ and R₂ independently represent C₁-C₄ alkyl, C₃-C₄ alkenyl or C₁-C₄ fluoroalkyl,
in a polar aprotic solvent in the presence of cesium carbonate (Cs₂CO₃) to form a carbamate of formula (V) [chemical structure]
wherein R, R₁ and R₂ are as previously defined, and
b) contacting the carbamate of formula (V) with a diboron compound of formula (VI) [chemical structure]
wherein R₃ₐ and R₄ₐ are as previously defined,
in a polar aprotic solvent in the presence of a palladium catalyst and an alkali metal or alkaline earth metal acetate.

8. A process for preparing a boronic ester of the formula (IIIa) [chemical structure]
wherein
R, R₁ and R₂ independently represent C₁-C₄ alkyl, C₃-C₄ alkenyl or C₁-C₄ fluoroalkyl, and
R₃ₐ and R₄ₐ independently represent C₁-C₄ alkyl, or when taken together form an ethylene or propylene group optionally substituted with from one to four CH₃ groups,
which comprises contacting a boronate substituted phenyl isocyanate of formula (VII) [chemical structure]
wherein
R₃ₐ and R₄ₐ independently represent C₁-C₄ alkyl, or when taken together form an ethylene or propylene group optionally substituted with from one to four CH₃ groups,
with a tetrahydropyran-2-ol of formula (IV) [chemical structure]
wherein
R, R₁ and R₂ independently represent C₁-C₄ alkyl, C₃-C₄ alkenyl or C₁-C₄ fluoroalkyl,
in a polar aprotic solvent in the presence of Cs₂CO₃.

9. A substituted triazole of formula (II) [chemical structure]
wherein
Y represents Cl, Br, I, OSO₂CF₃, OSO₂CH₃ or OSO₂C₆H₄CH₃, and
Z represents a furanyl, phenyl, pyridazinyl, pyridyl, pyrimidinyl or thienyl group, unsubstituted or substituted with one or more substituents independently selected from F, Cl, C₁-C₆ alkyl, C₁-C₆ haloalkyl, C₁-C₆ haloalkoxy or C₁-C₆ haloalkylthio.

10. A process for preparing a substituted triazole of formula (IIa) [chemical structure]
wherein
Z represents a furanyl, phenyl, pyridazinyl, pyridyl, pyrimidinyl or thienyl group, unsubstituted or substituted with one or more substituents independently selected from F, Cl, C₁-C₆ alkyl, C₁-C₆ haloalkyl, C₁-C₆ haloalkoxy or C₁-C₆ haloalkylthio,
which comprises contacting 3-bromo-1H-1,2,4-triazole [chemical structure]
with a brominated or iodinated furanyl, phenyl, pyridazinyl, pyridyl, pyrimidinyl or thienyl compound, unsubstituted or substituted with one or more substituents independently selected from F, Cl, C₁-C₆ alkyl, C₁-C₆ haloalkyl, C₁-C₆ haloalkoxy or C₁-C₆ haloalkylthio of one of the following formulas [chemical structure]
wherein
L represents Br or I,
X independently represents F, Cl, C₁-C₆ alkyl, C₁-C₆ haloalkyl, C₁-C₆ haloalkoxy or C₁-C₆ haloalkylthio,
m=0, 1, 2 or 3,
n=0, 1, 2, 3 or 4, and
p=0, 1, 2, 3, 4 or 5,
in a polar aprotic solvent in the presence of a catalytic amount of a copper catalyst and at least one equivalent of an inorganic base at a temperature from ambient to about 120° C.

11. A process for preparing a substituted triazole of formula (II) [chemical structure]
wherein
Y represents Cl, Br, I, OSO₂CF₃, OSO₂CH₃ or OSO₂C₆H₄CH₃, and
Z represents a furanyl, phenyl, pyridazinyl, pyridyl, pyrimidinyl or thienyl group, unsubstituted or substituted with one or more substituents independently selected from F, Cl, C₁-C₆ alkyl, C₁-C₆ haloalkyl, C₁-C₆ haloalkoxy or C₁-C₆ haloalkylthio,
which comprises
a) contacting a hydrazine hydrochloride of the formula
Z—NH—NH₂.HCl
wherein
Z represents a furanyl, phenyl, pyridazinyl, pyridyl, pyrimidinyl or thienyl group, unsubstituted or substituted with one or more substituents independently selected from F, Cl, C₁-C₆ alkyl, C₁-C₆ haloalkyl, C₁-C₆ haloalkoxy or C₁-C₆ haloalkylthio,
with urea in an aprotic organic solvent with a boiling point greater than 100° C. in the presence of a catalytic amount of an organic sulfonic acid at a temperature from about 100° C. to about 150° C.,
b) further contacting the reaction mixture from step a) with a C₁-C₄ alkyl orthoformate and a catalytic amount of chlorosulfonic acid at a temperature from about 60° C. to about 100° C. to provide a substituted 1-H-1,2,4-triazol-3-ol of formula (VIII) [chemical structure]
wherein Z is as previously defined, and
c) converting the hydroxyl group of the triazole to a Cl, Br, I, OSO₂CF₃, OSO₂CH₃ or OSO₂C₆H₄CH₃.
